SEQUENCE LISTING (80 sequences)

SEQ ID NO: 1 - RNA, 22 nt, synthetic construct
  misc_feature 1..22: note = Biomarker
  source 1..22: mol_type = other RNA
tggaatgtaaagaagtatgtat

SEQ ID NO: 2 - RNA, 22 nt, synthetic construct
  misc_feature 1..22: note = Biomarker
  source 1..22: mol_type = other RNA
tggaatgtaaggaagtatgtat

SEQ ID NO: 3 - RNA, 21 nt, synthetic construct
  misc_feature 1..21: note = Biomarker
  source 1..21: mol_type = other RNA
tggaatgtaaagaagtatgta

SEQ ID NO: 4 - RNA, 20 nt, synthetic construct
  misc_feature 1..20: note = Biomarker
  source 1..20: mol_type = other RNA
tggaatgtaaagaagtatgt

SEQ ID NO: 5 - RNA, 11 nt, synthetic construct
  misc_feature 1..11: note = Biomarker
  source 1..11: mol_type = other RNA
tggaatgtaaa

SEQ ID NO: 6 - RNA, 21 nt, synthetic construct
  misc_feature 1..21: note = Biomarker
  source 1..21: mol_type = other RNA
atgacctatgaattgacagac

SEQ ID NO: 7 - RNA, 23 nt, synthetic construct
  misc_feature 1..23: note = Biomarker
  source 1..23: mol_type = other RNA
caaagtgcttacagtgcaggtag

SEQ ID NO: 8 - RNA, 22 nt, synthetic construct
  misc_feature 1..22: note = Biomarker
  source 1..22: mol_type = other RNA
tagcaccatctgaaatcggtta

SEQ ID NO: 9 - RNA, 22 nt, synthetic construct
  misc_feature 1..22: note = Biomarker
  source 1..22: mol_type = other RNA
tagcagcacatcatggtttaca

SEQ ID NO: 10 - RNA, 22 nt, synthetic construct
  misc_feature 1..22: note = Biomarker
  source 1..22: mol_type = other RNA
tgaggtagtaggttgtatggtt

SEQ ID NO: 11 - RNA, 22 nt, synthetic construct
  misc_feature 1..22: note = Biomarker
  source 1..22: mol_type = other RNA
ctttcagtcggatgtttacagc

SEQ ID NO: 12 - RNA, 21 nt, synthetic construct
  misc_feature 1..21: note = Biomarker
  source 1..21: mol_type = other RNA
ttcacagtggctaagttccgc

SEQ ID NO: 13 - RNA, 22 nt, synthetic construct
  misc_feature 1..22: note = Biomarker
  source 1..22: mol_type = other RNA
tggaatgtaaagaagtatgtat

SEQ ID NO: 14 - RNA, 21 nt, synthetic construct
  misc_feature 1..21: note = Biomarker
  source 1..21: mol_type = other RNA
ctgacctatgaattgacagac

SEQ ID NO: 15 - RNA, 23 nt, synthetic construct
  misc_feature 1..23: note = Biomarker
  source 1..23: mol_type = other RNA
caaagtgctcacagtgcaggtag

SEQ ID NO: 16 - RNA, 22 nt, synthetic construct
  misc_feature 1..22: note = Biomarker
  source 1..22: mol_type = other RNA
tagcaccatttgaaatcggtta

SEQ ID NO: 17 - RNA, 22 nt, synthetic construct
  misc_feature 1..22: note = Biomarker
  source 1..22: mol_type = other RNA
tagcagcacataatggtttaca

SEQ ID NO: 18 - RNA, 22 nt, synthetic construct
  misc_feature 1..22: note = Biomarker
  source 1..22: mol_type = other RNA
tgaggtagtaggttgtgtggtt

SEQ ID NO: 19 - RNA, 22 nt, synthetic construct
  misc_feature 1..22: note = Biomarker
  source 1..22: mol_type = other RNA
ctttcagtcggatgtttgcagc

SEQ ID NO: 20 - RNA, 21 nt, synthetic construct
  misc_feature 1..21: note = Biomarker
  source 1..21: mol_type = other RNA
ttcacagtggctaagttctgc

SEQ ID NO: 21 - RNA, 22 nt, synthetic construct
  misc_feature 1..22: note = Biomarker
  source 1..22: mol_type = other RNA
tggaatgtaaagaagtatgaat

SEQ ID NO: 22 - RNA, 22 nt, synthetic construct
  misc_feature 1..22: note = Biomarker
  source 1..22: mol_type = other RNA
tggaatgtaaagaagtatggat

SEQ ID NO: 23 - RNA, 22 nt, synthetic construct
  misc_feature 1..22: note = Biomarker
  source 1..22: mol_type = other RNA
tggaatgtaaagaagtatgcat

SEQ ID NO: 24 - RNA, 17 nt, synthetic construct
  misc_feature 1..17: note = Biomarker
  source 1..17: mol_type = other RNA
tggaatgtaaagaagta

SEQ ID NO: 25 - RNA, 17 nt, synthetic construct
  misc_feature 1..17: note = Biomarker
  source 1..17: mol_type = other RNA
tggaatgtaaggaagta

SEQ ID NO: 26 - RNA, 11 nt, synthetic construct
  misc_feature 1..11: note = Biomarker
  source 1..11: mol_type = other RNA
tggaatgtaag

SEQ ID NO: 27 - RNA, 12 nt, synthetic construct
  misc_feature 1..12: note = Biomarker
  source 1..12: mol_type = other RNA
ctgacctatgaa

SEQ ID NO: 28 - RNA, 10 nt, synthetic construct
  misc_feature 1..10: note = Biomarker
  source 1..10: mol_type = other RNA
caaagtgctc

SEQ ID NO: 29 - RNA, 10 nt, synthetic construct
  misc_feature 1..10: note = Biomarker
  source 1..10: mol_type = other RNA
tagcaccatt

SEQ ID NO: 30 - RNA, 16 nt, synthetic construct
  misc_feature 1..16: note = Biomarker
  source 1..16: mol_type = other RNA
tggaatgtaaggaagt

SEQ ID NO: 31 - RNA, 19 nt, synthetic construct
  misc_feature 1..19: note = Biomarker
  source 1..19: mol_type = other RNA
tagcagcacataatggttt

SEQ ID NO: 32 - RNA, 17 nt, synthetic construct
  misc_feature 1..17: note = Biomarker
  source 1..17: mol_type = other RNA
tgaggtagtaggttgtg

SEQ ID NO: 33 - RNA, 18 nt, synthetic construct
  misc_feature 1..18: note = Biomarker
  source 1..18: mol_type = other RNA
ctttcagtcggatgtttg

SEQ ID NO: 34 - RNA, 19 nt, synthetic construct
  misc_feature 1..19: note = Biomarker
  source 1..19: mol_type = other RNA
ttcacagtggctaagttct

SEQ ID NO: 35 - RNA, 19 nt, synthetic construct
  misc_feature 1..19: note = Biomarker
  source 1..19: mol_type = other RNA
tggaatgtaaagaagtatg

SEQ ID NO: 36 - RNA, 12 nt, synthetic construct
  misc_feature 1..12: note = Biomarker
  source 1..12: mol_type = other RNA
atgacctatgaa

SEQ ID NO: 37 - RNA, 10 nt, synthetic construct
  misc_feature 1..10: note = Biomarker
  source 1..10: mol_type = other RNA
caaagtgctv

SEQ ID NO: 38 - RNA, 10 nt, synthetic construct
  misc_feature 1..10: note = Biomarker
  source 1..10: mol_type = other RNA
tagcaccatc

SEQ ID NO: 39 - RNA, 16 nt, synthetic construct
  misc_feature 1..16: note = Biomarker
  source 1..16: mol_type = other RNA
tggaatgtaaagaagt

SEQ ID NO: 40 - RNA, 19 nt, synthetic construct
  misc_feature 1..19: note = Biomarker
  source 1..19: mol_type = other RNA
tagcagcacatcatggttt

SEQ ID NO: 41 - RNA, 17 nt, synthetic construct
  misc_feature 1..17: note = Biomarker
  source 1..17: mol_type = other RNA
tgaggtagtaggttgta

SEQ ID NO: 42 - RNA, 18 nt, synthetic construct
  misc_feature 1..18: note = Biomarker
  source 1..18: mol_type = other RNA
ctttcagtcggatgttta

SEQ ID NO: 43 - RNA, 19 nt, synthetic construct
  misc_feature 1..19: note = Biomarker
  source 1..19: mol_type = other RNA
ttcacagtggctaagttcc

SEQ ID NO: 44 - RNA, 19 nt, synthetic construct
  misc_feature 1..19: note = Biomarker
  source 1..19: mol_type = other RNA
tggaatgtaaagaagtttg

SEQ ID NO: 45 - RNA, 19 nt, synthetic construct
  misc_feature 1..19: note = Biomarker
  source 1..19: mol_type = other RNA
ctgacctatgaattgacag

SEQ ID NO: 46 - RNA, 18 nt, synthetic construct
  misc_feature 1..18: note = Biomarker
  source 1..18: mol_type = other RNA
ctgacctatgaattgaca

SEQ ID NO: 47 - RNA, 17 nt, synthetic construct
  misc_feature 1..17: note = Biomarker
  source 1..17: mol_type = other RNA
ctgacctatgaattgac

SEQ ID NO: 48 - RNA, 16 nt, synthetic construct
  misc_feature 1..16: note = Biomarker
  source 1..16: mol_type = other RNA
ctgacctatgaattga

SEQ ID NO: 49 - RNA, 15 nt, synthetic construct
  misc_feature 1..15: note = Biomarker
  source 1..15: mol_type = other RNA
ctgacctatgaattg

SEQ ID NO: 50 - RNA, 14 nt, synthetic construct
  misc_feature 1..14: note = Biomarker
  source 1..14: mol_type = other RNA
ctgacctatgaatt

SEQ ID NO: 51 - RNA, 13 nt, synthetic construct
  misc_feature 1..13: note = Biomarker
  source 1..13: mol_type = other RNA
ctgacctatgaat

SEQ ID NO: 52 - RNA, 11 nt, synthetic construct
  misc_feature 1..11: note = Biomarker
  source 1..11: mol_type = other RNA
ctgacctatga

SEQ ID NO: 53 - RNA, 10 nt, synthetic construct
  misc_feature 1..10: note = Biomarker
  source 1..10: mol_type = other RNA
ctgacctatg

SEQ ID NO: 54 - RNA, 21 nt, synthetic construct
  misc_feature 1..10: note = Biomarker
  source 1..21: mol_type = other RNA
tagcaccatttgaaatcggtt

SEQ ID NO: 55 - RNA, 20 nt, synthetic construct
  misc_feature 1..20: note = Biomarker
  source 1..20: mol_type = other RNA
tagcaccatttgaaatcggt

SEQ ID NO: 56 - RNA, 19 nt, synthetic construct
  misc_feature 1..19: note = Biomarker
  source 1..19: mol_type = other RNA
tagcaccatttgaaatcgg

SEQ ID NO: 57 - RNA, 18 nt, synthetic construct
  misc_feature 1..18: note = Biomarker
  source 1..18: mol_type = other RNA
tagcaccatttgaaatcg

SEQ ID NO: 58 - RNA, 17 nt, synthetic construct
  misc_feature 1..17: note = Biomarker
  source 1..17: mol_type = other RNA
tagcaccatttgaaatc

SEQ ID NO: 59 - RNA, 16 nt, synthetic construct
  misc_feature 1..16: note = Biomarker
  source 1..16: mol_type = other RNA
tagcaccatttgaaat

SEQ ID NO: 60 - RNA, 15 nt, synthetic construct
  misc_feature 1..15: note = Biomarker
  source 1..15: mol_type = other RNA
tagcaccatttgaaa

SEQ ID NO: 61 - RNA, 14 nt, synthetic construct
  misc_feature 1..14: note = Biomarker
  source 1..14: mol_type = other RNA
tagcaccatttgaa

SEQ ID NO: 62 - RNA, 13 nt, synthetic construct
  misc_feature 1..13: note = Biomarker
  source 1..13: mol_type = other RNA
tagcaccatttga

SEQ ID NO: 63 - RNA, 12 nt, synthetic construct
  misc_feature 1..12: note = Biomarker
  source 1..12: mol_type = other RNA
tagcaccatttg

SEQ ID NO: 64 - RNA, 11 nt, synthetic construct
  misc_feature 1..11: note = Biomarker
  source 1..11: mol_type = other RNA
tagcaccattt

SEQ ID NO: 65 - RNA, 15 nt, synthetic construct
  misc_feature 1..11: note = Biomarker
  source 1..15: mol_type = other RNA
tggaatgtaaggaag

SEQ ID NO: 66 - RNA, 14 nt, synthetic construct
  misc_feature 1..14: note = Biomarker
  source 1..14: mol_type = other RNA
tggaatgtaaggaa

SEQ ID NO: 67 - RNA, 13 nt, synthetic construct
  misc_feature 1..13: note = Biomarker
  source 1..13: mol_type = other RNA
tggaatgtaagga

SEQ ID NO: 68 - RNA, 12 nt, synthetic construct
  misc_feature 1..12: note = Biomarker
  source 1..12: mol_type = other RNA
tggaatgtaagg

SEQ ID NO: 69 - RNA, 18 nt, synthetic construct
  misc_feature 1..12: note = Biomarker
  source 1..18: mol_type = other RNA
tagcagcacataatggtt

SEQ ID NO: 70 - RNA, 17 nt, synthetic construct
  misc_feature 1..11: note = Biomarker
  source 1..17: mol_type = other RNA
tagcagcacataatggt

SEQ ID NO: 71 - RNA, 16 nt, synthetic construct
  misc_feature 1..16: note = Biomarker
  source 1..16: mol_type = other RNA
tagcagcacataatgg

SEQ ID NO: 72 - RNA, 15 nt, synthetic construct
  misc_feature 1..15: note = Biomarker
  source 1..15: mol_type = other RNA
tagcagcacataatg

SEQ ID NO: 73 - RNA, 14 nt, synthetic construct
  misc_feature 1..14: note = Biomarker
  source 1..14: mol_type = other RNA
tagcagcacataat

SEQ ID NO: 74 - RNA, 13 nt, synthetic construct
  misc_feature 1..13: note = Biomarker
  source 1..13: mol_type = other RNA
tagcagcacataa

SEQ ID NO: 75 - RNA, 12 nt, synthetic construct
  misc_feature 1..12: note = Biomarker
  source 1..12: mol_type = other RNA
tagcagcacata

SEQ ID NO: 76 - RNA, 18 nt, synthetic construct
  misc_feature 1..13: note = Biomarker
  source 1..18: mol_type = other RNA
tgaggtagtaggttgtgt

SEQ ID NO: 77 - RNA, 19 nt, synthetic construct
  misc_feature 1..12: note = Biomarker
  source 1..19: mol_type = other RNA
ctttcagtcggatgtttgc

SEQ ID NO: 78 - RNA, 20 nt, synthetic construct
  misc_feature 1..18: note = Biomarker
  source 1..20: mol_type = other RNA
ttcacagtggctaagttctg

SEQ ID NO: 79 - RNA, 18 nt, synthetic construct
  misc_feature 1..18: note = Biomarker
  source 1..18: mol_type = other RNA
tggaatgtaaagaagtat

SEQ ID NO: 80 - RNA, 17 nt, synthetic construct
  misc_feature 1..17: note = Biomarker
  source 1..17: mol_type = other RNA
tggaatgtaaagaagta

(19) United States
(12) Patent Application Publication
(10) Pub. No.: US 2026/0002221 A1
(43) Pub. Date: Jan. 1, 2026

(54) SAMPLE IDENTIFICATION METHOD AND BIOMARKER

(71) Applicant: KABUSHIKI KAISHA TOSHIBA, Kawasaki-shi (JP)

(72) Inventors: Tomomi ANDO, Ota (JP); Mitsuko ISHIHARA, Setagaya (JP); Yoshitake SANO, Adachi (JP)

(73) Assignee: KABUSHIKI KAISHA TOSHIBA, Kawasaki-shi (JP)

(21) Appl. No.: 19/308,604

(22) Filed: Aug. 25, 2025

Related U.S. Application Data
(63) Continuation of application No. PCT/JP2024/023960, filed on Jul. 2, 2024.

(30) Foreign Application Priority Data
Sep. 19, 2023 (JP) 2023-150929

(51) Int. Cl. C12Q 1/6886

(52) U.S. Cl. CPC C12Q 1/6886; C12Q 2600/178

(57) ABSTRACT

A sample identification method according to an embodiment includes setting a first reference sequence that includes a first sequence having a single base substitution site, the single base substitution site being a first base, and a corresponding second reference sequence, outputting the number of first short-chain nucleic acids having the first reference sequence and the number of second short-chain nucleic acids having the second reference sequence, calculating a ratio, obtaining a magnitude relationship between the ratio and a threshold value, and determining, from the number of sequences in which the ratio is greater than the threshold value, whether a subject from whom the sample is derived has the disease or at risk of developing the disease.

### CROSS-REFERENCE TO RELATED APPLICATIONS

This application is a Continuation Application of PCT Application No. PCT/JP2024/023960, filed Jul. 2, 2024 and based upon and claiming the benefit of priority from Japanese Patent Application No. 2023-150929, filed Sep. 19, 2023, the entire contents of all of which are incorporated herein by reference.

### REFERENCE TO A SEQUENCE LISTING

A Sequence Listing, submitted as an XML file and compliant with WIPO Standard ST.26, forms part of the present application. The Sequence Listing is identified as follows: File name “559452US_ST26.xml,” created on Aug. 22, 2025, with a size of 101,083 bytes.

### FIELD

Embodiments of the present invention relate to a sample identification method and a biomarker.

### BACKGROUND

A test method in which a cancer patient and a healthy individual are identified by analyzing a nucleic acid separated from a body fluid that can be easily collected is known. For example, an identification system using miRNA (microRNA) has been widely verified. The miRNA is a single-stranded nucleic acid of about 17 to 25 bases, and has been revealed to have a function of regulating gene expression. It has also been reported that its type and expression level of various diseases are changed from the initial stage. For example, in the cancer patient, various miRNAs are used as cancer markers. This is based on the finding that the amount thereof present in a patient is increased or decreased compared to a healthy individual.

In the test using a miRNA amount or a miRNA concentration as an index, the obtained result or tendency tends to be different between specimens or data sets. As a result, a variation between the data and the results increases, and the test performance tends to be unstable.

### BRIEF DESCRIPTION OF THE DRAWINGS

FIG. 1 is a scheme diagram illustrating a first embodiment.

FIG. 2 is an image diagram of a result obtained by the first embodiment.

FIG. 3 is a scheme diagram illustrating a second embodiment.

FIG. 4 is an image diagram of the results obtained by the second and third embodiments.

FIG. 5 is a scheme diagram illustrating a procedure of Example 1.

FIG. 6 is a graph illustrating a result of Example 2.

FIG. 7 is a graph illustrating the result of Example 2.

FIG. 8 is a graph illustrating the result of Example 2.

FIG. 9 is a diagram illustrating a result of Example 3.

FIG. 10 is a diagram illustrating a result of Example 4.

FIG. 11 is a diagram illustrating a result of Example 5.

### DETAILED DESCRIPTION

In general, according to one embodiment, a method for identifying a sample is a method for identifying a sample as to whether or not a subject from whom the sample is derived has a disease.

The method comprises: setting a set or a plurality of sets of a first reference sequence that includes a first sequence having a single base substitution site that is suggested to have a relationship with a target disease based on a predetermined criterion, the single base substitution site being a first base, and a second reference sequence that corresponds to the first reference sequence, includes the first sequence, and contains a second base having the single base substitution site that is different from the first base; outputting, with respect to a short-chain nucleic acid group contained in the sample, the number of first short-chain nucleic acids (the number of ID-2) having a sequence having a length of 10 bases or more and having the single base substitution site so as to be prefix matched with the first reference sequence from the 5′-end thereof, and the number of second short-chain nucleic acids (the number of ID-1) having a sequence having a length of 10 bases or more and having the single base substitution site so as to be prefix matched with the second reference sequence from the 5′-end thereof; calculating a ratio R=(the number of ID-1/(the number of ID-1+the number of ID-2)) for each corresponding sequence; comparing a value of the ratio R with a predetermined threshold value to obtain a magnitude relationship between the value of the ratio R and the predetermined threshold value; and determining, from the number of sequences in which the value of the ratio R is greater than the threshold value, whether or not a subject from whom the sample is derived has the disease or at risk of developing the disease.

Hereinafter, embodiments will be described with reference to the accompanying drawings. Note that, in each of the embodiments, substantially the same constituents are denoted by the same reference numerals, and the description thereof may be partially omitted. The drawings are schematic, and a relationship between a thickness and a planar dimension of each part may be actually different from a ratio of the thickness of each part.

### First Embodiment

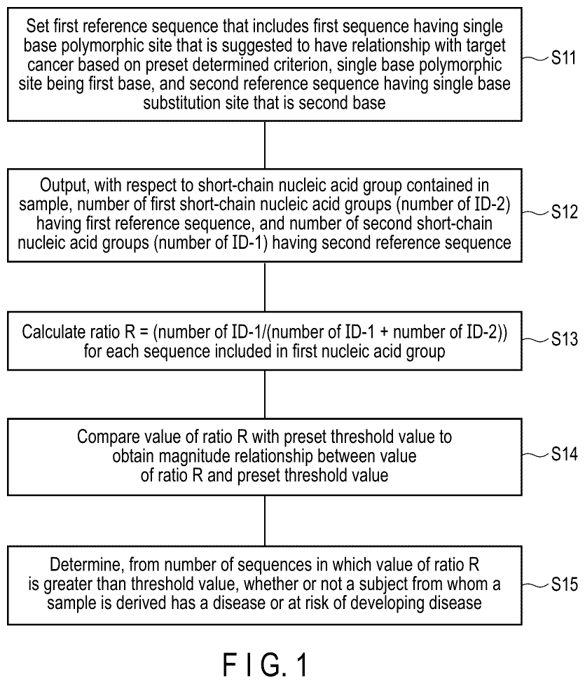

A first embodiment is a method for identifying a sample as to whether or not a subject from whom the sample is derived has a disease. The method is as illustrated in FIG. 1. In S11, a reference sequence set is set. A reference sequence set includes a first reference sequence and a second reference sequence corresponding thereto. The first reference sequence is represented by a first sequence having a single base substitution site whose relationship with a target disease is suggested based on a predetermined criterion. The second reference sequence is a sequence corresponding to the first reference sequence. That is, the second reference sequence is the same as the first sequence except for the type of base of the single base substitution site. In other words, the first reference sequence and the second reference sequence are common to the first sequence except for the single base substitution site. Here, one or a plurality of reference sequence sets may be set.

In S12, regarding a short-chain nucleic acid group contained in the sample, the number of first short-chain nucleic acids (the number of ID-2) and the number of second short-chain nucleic acids (the number of ID-1) are output. The first short-chain nucleic acid is a short-chain nucleic acid having a sequence in which the full length of the first reference sequence and/or a base on the 3′-end side of the first reference sequence is deleted at the 5′-end thereof. The first reference sequence in which the base on the 3′-end side is deleted can be a sequence having the single base substitution site, and having a length of 8 bases or more, for example, a length of 9 bases or more, or a length of 10 bases or more, for example, a sequence of at least 10 bases, so as to be prefix matched from the 5′-end. The sequence in which the base on the 3′-end side of the first reference sequence is deleted may be all sequences of 8 bases or more, 9 bases or more, or 10 bases or more in length obtained by deleting one base at the 3′-end in order from the full-length sequence. In other words, a short-chain nucleic acid having a sequence in which a full length of the first reference sequence and/or a base on the 3′-end side of the first reference sequence is deleted is a first short-chain nucleic acid, and a plurality of different base sequences can correspond to the first short-chain nucleic acid. In addition, in the case of having a sequence in which a base on the 3′-end side of the first reference sequence is deleted, the first short-chain nucleic acid may have all sequences different depending on the number of bases deleted, or may have at least one type of sequence. The first short-chain nucleic acid may have any sequence longer than the length of the base to be deleted. The same applies to the second short-chain nucleic acid. Here, when the length of the reference sequence is too short, the identification accuracy may be lowered. For example, in the case of the miRNA, the base length is preferably longer than a seed sequence. The length of the short- chain nucleic acid detected in the sample is not particularly limited.

Subsequently, in S13, a ratio R=(the number of ID-1/(the number of ID-1+the number of ID-2)) is calculated for each corresponding sequence. Calculating for each corresponding sequence means calculating for each reference sequence set or for each type or length of the corresponding sequence. In S14, the value of the ratio R is compared with a predetermined threshold value to obtain a magnitude relationship therebetween. Subsequently, in S15, based on the number of sequences in which the value of the ratio R is greater than the threshold value, it is determined whether or not a subject from whom the sample is derived has a disease or at risk of developing a disease.

The “sample” may be a cell, tissue, and/or liquid collected from a subject, or a mixture thereof, or a treated product obtained by appropriately treating them. The “sample” may be a body fluid extracted externally from a subject. When the sample is a body fluid, for example, the sample may be serum or plasma, or other body fluids, for example, blood, white blood cell interstitial fluid, urine, feces, sweat, saliva, oral mucosa, intranasal mucosa, nasal mucus, pharyngeal mucosa, sputum, digestive juice, gastric juice, lymph, cerebrospinal fluid, tear fluid, breast milk, amniotic fluid, semen, vaginal fluid, or a mixture thereof. For example, the sample may be just collected from a subject, cultured, preserved in a desired procedure, or may be a supernatant thereof obtained after maintaining the sample in a desired liquid. For example, it is preferable to use a body fluid such as blood, serum, or plasma as a sample because it is easy to collect.

The method of the embodiment analyzes a short-chain nucleic acid group contained in the sample as described above. The “short-chain nucleic acid group” can be, for example, RNA. For example, a preferred example is mRNA, non-coding RNA (ncRNA), housekeeping ncRNA, tRNA, small ncRNA, miRNA, piRNA, tsRNA, or IncRNA. For example, a more preferred example is miRNA.

The “identification” of the sample can be to identify whether the subject from which a sample to be tested is derived is suffering from a disease, is at risk of developing a disease, or is at high risk of developing a disease.

The “subject” may be plants and animals from which a sample is to be collected. For example, the subject may be a mammal, including a human, or a non-human mammal. For example, the subject may be an animal belonging to a primate such as a human or a monkey, a rodent such as a mouse, a rat, or a guinea pig, a companion animal such as a dog, a cat, or a rabbit, a livestock animal such as a horse, a cow, or a pig, or a display animal.

For example, a morbidity state of such a subject can be tested by using a sample identification method. In other words, the sample identification method can also be used to diagnose a subject. The results obtained by the sample identification method may assist a doctor in diagnosis. Alternatively, the sample identification method can be used to assist in screening a disease, such as a medical examination.

In the method, the information on RNA for the sample may be acquired after the RNA is extracted from the sample. The method for extracting RNA may be a method known per se, and for example, it is also possible to use a commercially available kit.

Here, the “disease” may be any disease to be detected, and may be any disease for which a certain degree of relationship with the reference sequence is predicted or tends to be related. For example, such a disease is cancer. Here, the cancer includes cancer at any stage, and includes, for example, a state in which cancer remains in the organ where it originated, a state in which cancer has spread to surrounding tissues, a state in which cancer has metastasized to the lymph node, a state in which cancer has metastasized to further distant organs, and the like. For example, the cancer may be at least one cancer selected from the group consisting of breast cancer, colorectal cancer, lung cancer, stomach cancer, pancreatic cancer, cervical cancer, uterine cancer, ovarian cancer, sarcoma, prostate cancer, bile duct cancer, bladder cancer, esophageal cancer, liver cancer, brain tumor, and kidney cancer.

An example of the “first reference sequence including a first sequence having a single base substitution site whose relationship with a target disease is suggested based on a predetermined criterion, in which the single base substitution site is a first base” is shown in Table 1.

TABLE 1
Example) 11^{th} sequence of No. 133
hsa-miR-1-3p: A → G:
TGGAATGTAA (A/G) GAAGTATGTAT

| Prepared | 133_WT_full | 133_mu_full |
|---|---|---|
| sequence | (or −5, min) | (or −5, min) |
| Full-length | TGGAATGTAAA | TGGAATGTAAGG |
| sequence | GAAGTATGTAT | AAGTATGTAT |
| | (SEQ ID NO: 1) | (SEQ ID NO: 2) |
| bp deletion | TGGAATGTAAA | TGGAATGTAAGG |
| from end | GAAGTA | AAGTA |
| | (SEQ ID NO: 24) | (SEQ ID NO: 25) |
| Deletion to | TGGAATGTAAA | TGGAATGTAAG |
| mutation | (SEQ ID NO: 5) | (SEQ ID NO: 26) |
| site | | |

The example shown in Table 1 is an example of a short-chain sequence suggesting a relationship between the base species of the substitution site and pancreatic cancer. The short-chain sequence is referred to as “hsa-miR-1-3p”, the full length of hsa-miR-1-3p is 22 bases, and the short-chain sequence is an RNA sequence including a sequence represented by TGGAATGTAA(A/G)GAAGTATGTAT in the 5′ to 3′ direction. The 11^{th} from the 5′-end of the sequence is a single base substitution site. Here, two types of bases (A and G) that can enter the substitution site are simultaneously described in parentheses. A wild-type base is adenine (A). That is, a wild-type sequence is TGGAATGTAA(A)GAAGTATGTAT (SEQ ID NO: 1).

The base of the single base substitution site in the second reference sequence, which corresponds to such a first reference sequence, is guanine (G) that is a second base different from A that is the wild-type. That is, the second reference sequence is, for example, TGGAATGTAA(G)GAAGTATGTAT (SEQ ID NO: 2). The first reference sequence and the second reference sequence have a common sequence other than the single base substitution site. In addition, as shown in Table 2, an additional second reference sequence corresponding to the first reference sequence may be a sequence having a base different from adenine (A), which is a base of the single base substitution site in the first reference sequence, that is, cytosine (C) and thymine (T) in the single base substitution site. These additional second reference sequences are sequences that have a common sequence other than the single base substitution site with the first reference sequence and the previous second reference sequence.

As shown in Table 2, the first short-chain nucleic acid (ID-2) has a sequence having a length of 10 bases or more and having the single base substitution site so that the first reference sequence is prefix matched from the 5′-end thereof. Here, SEQ ID NO: 1 represents a full-length sequence of the first reference sequence. That is, in the case of the sequence, the deletion at the 3′-end is 0. In SEQ ID NO: 1, the single base substitution site is at the 11^{th} base from the 5′-end and at the 12^{th} base from the 3′-end. Here, “having a sequence having a length of 10 bases or more and having the single base substitution site so as to be prefix matched from the 5′-end thereof” can be read as “including any position from the single base substitution site to the 3′-end”.

That is, Table 2 shows sequences in which the bases are deleted one by one from the 3′-end side in the full-length sequence of the first reference sequence up to the base immediately before the single base substitution site. The first short-chain nucleic acid (ID-2) has, as part of a sequence of varying length, a sequence including any of the full-length sequence-0, full-length sequence-1, full-length sequence-2, full-length sequence-3, . . . , full-length sequence-n so as to be prefix matched from the 5′-end thereof. Here, n is the number of bases from the 3′-end of the first reference sequence to immediately before the single base substitution site. Note that, although not applicable in this case, in other cases, for example, when a single base substitution site is located at a position less than 10 bases from the 5′-end side, n may be the maximum integer so that the length of the shortened reference sequence due to deletion of the 3′-end is 10 bases or more. Referring to Table 2, for example, when the first reference sequence is the sequence set forth in SEQ ID NO: 1, n is 11. That is, in this case, the first short-chain nucleic acid (ID-2) can include a nucleic acid having a full-length sequence 0, a full-length sequence-1, a full-length sequence-2, a full-length sequence-3, a full-length sequence-4, a full-length sequence-5, a full-length sequence-6, a full-length sequence-7, a full-length sequence-8, a full-length sequence-9, a full-length sequence-10, or a full-length sequence-11 as one short-chain nucleic acid, and a nucleic acid having a full-length sequence 0, a full-length sequence-1, a full-length sequence-2, a full-length sequence-3, a full-length sequence-4, a full-length sequence-5, a full-length sequence-6, a full-length sequence-7, a full-length sequence-8, a full-length sequence-9, a full-length sequence-10, or a full-length sequence-11 as a part of one short-chain nucleic acid on the 5′-end thereof. In other words, the first short-chain nucleic acid (ID-2) may have all sequences detectable using the full-length sequence 0, the full-length sequence-1, the full-length sequence-2, the full-length sequence-3, the full-length sequence-4, the full-length sequence-5, the full-length sequence-6, the full-length sequence-7, the full-length sequence-8, the full-length sequence-9, the full-length sequence-10, or the full-length sequence-11 as an index. The same applies to the second short-chain nucleic acid (ID-1). What is output in (S12) are the number of first short-chain nucleic acids (ID-2) and the corresponding number of second short-chain nucleic acids (ID-1) in the sample.

Here, the “output” may have any form as long as the ratio R=(the number of ID-1/(the number of ID-1+the number of ID-2)) in the following (S13) can be calculated. For example, the number of ID-1 and the number of ID-2 by manual experimentation may be determined by a practitioner of the method, may be automatically derived by a reaction and/or analysis device, or may be used in combination. In addition, for example, these numbers may be actually output to an output unit such as a display, or may be temporarily held in a CPU or a calculation unit of a computer, for example. The “predetermined criterion” may be, for

example, AUC. For example, showing a specific AUC value, for example, an AUC value of 0.7 or more, may be used as a criterion. Here, the AUC is an index used to represent the accuracy of the test. For example, the AUC can be determined as follows using a graph. That is, the AUC is indicated as an area under an ROC curve when the ROC curve is created when a threshold value is continuously changed with sensitivity on the vertical axis and (1—specificity) on the horizontal axis as 0 to 1. Here, the “specificity” is a numerical value of 0 to 1 indicating a proportion of the test value less than or equal to the threshold value among the test values of a healthy group in a case where it is determined as positive when the test value exceeds a certain threshold value. It is shown that the closer the AUC is to 1, that is, the closer the curve is to the upper left corner, the higher the diagnostic accuracy of the diagnostic method is, and the closer the AUC is to 0.5, that is, the closer the curve is to the diagonal, the lower the diagnostic accuracy of the diagnostic method is.

In addition, here, the “test value of the healthy group” may be data obtained from a healthy individual. The “healthy individual” refers to an individual not suffering from cancer, and may be preferably an individual not suffering from a malignant neoplasm including cancer or a malignant tumor, that is, a “non-cancer patient”. The “predetermined criterion” may be determined in advance prior to the execution of the method on the basis of past data, or may be set simultaneously with the execution of the method on the basis of these data. For example, whether the origin is derived from a non-cancer patient or a cancer patient can be determined using a sample or a control sample whose origin is clear.

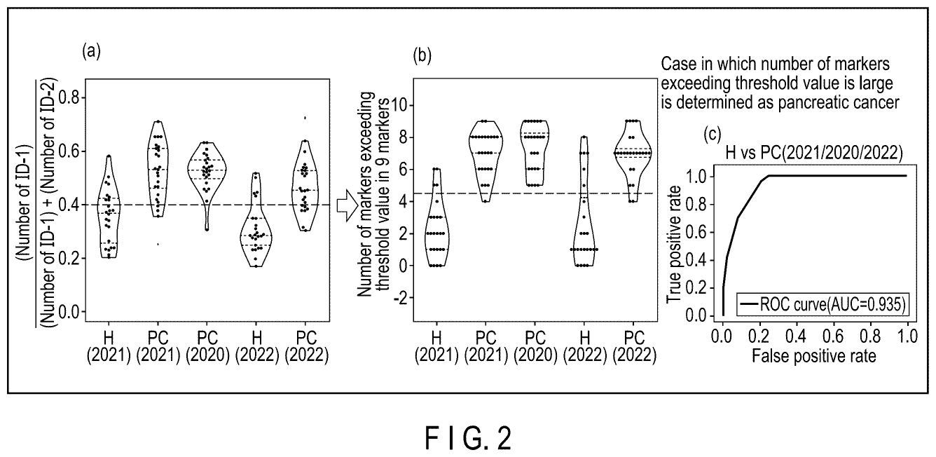

In (S13), a ratio R=(the number of ID-1/(the number of ID-1+the number of ID-2)) is calculated for each corresponding sequence. Using the magnitude of the ratio R as an index, it is possible to identify between a sample derived from a non-cancer patient and a sample derived from a cancer patient. In order to distinguish a sample derived from a non-cancer patient and a sample derived from a cancer patient, it is possible to use a predetermined threshold value using a control sample whose origin is clear. When the value of the ratio R of the sample to be identified is greater than a predetermined threshold value, it can be determined that the sample is from a subject having a disease or at risk of developing a disease. FIG. 2 illustrates an example of a graph showing the relationship between the ratio R, the sample derived from a non-cancer patient, the sample derived from a cancer patient, and the threshold value.

FIG. 2 illustrates an example of the results obtained using 9 types of base sequence sets in 5 different sample groups as markers, represented as the distribution of the ratio R. FIG. 2(a) is one of graphs created for each of the 9 types of markers. In the graph, the ratio R is plotted along the vertical axis, and sample groups H (2021) and H (2022) derived from two different non-cancer patient subject groups and sample groups PC (2020), PC (2021), and PC (2022) derived from three different pancreatic cancer patient groups are plotted along the horizontal axis. Data from each sample group were each spotted as one point per sample within a violin plot showing the distribution of the data. FIG. 2(b) illustrates the results of plotting the number of markers exceeding the threshold value on the vertical axis among the data obtained for each of 9 types of markers used.

This step corresponds to a step of counting

“the number of sequences in which the value of the ratio R is greater than the threshold value”. This step makes it possible to more clearly visualize a difference in data tendency between data from a sample group derived from a non-cancer patient and data from a sample group derived from a cancer patient using a threshold value. FIG. 2(c) is a result of plotting the data of FIG. 2(b). FIG. 2(c) is a graph of ROC curves between sample groups H (2021) and H (2022) derived from a non-cancer patient subject group and sample groups PC (2020), PC (2021), and PC (2022) derived from a pancreatic cancer patient group. The vertical axis of the graph represents a true positive rate, and the horizontal axis represents a false positive rate. In the case of this data, because of its AUC value is 0.935, it is an example of a graph showing that identification can be performed with high accuracy. In order to determine “from the number of sequences in which the value of the ratio R is greater than the threshold value”, the number of sequences in which the value of the ratio R is great is counted and compared with a threshold value set for the number of markers used to determine whether or not a subject from whom the sample is derived has a disease or at risk of developing a disease. For example, in FIG. 2(b), when the value of the ratio R is great in 5 or more of the 9 types of markers, it can be determined that the sample is derived from a subject having a disease or at risk of developing a disease.

According to the first embodiment, a method capable of identifying a sample derived from a patient with higher accuracy is provided.

### Second Embodiment

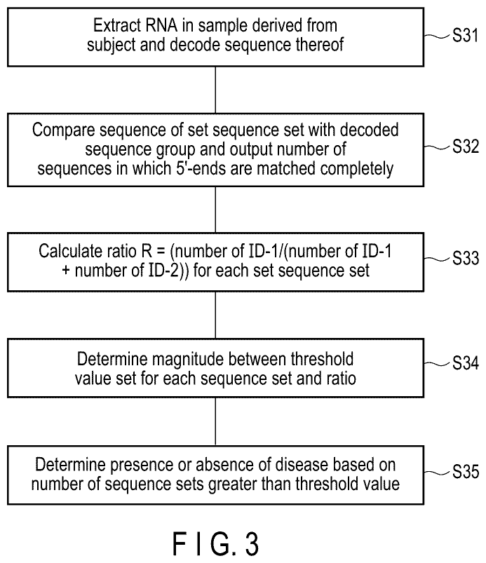

A second embodiment is another method for identifying a sample as to whether or not a subject from whom the sample is derived has a disease. This method will be described with reference to FIG. 3. First, in S31, RNA in a sample derived from a subject is extracted, and sequence group thereof are decoded. Subsequently, in S32, the sequence of the sequence set which are set in advance is compared with the sequence group decoded in S31, and the number of sequences in which 5′-ends are matched completely is output. Next, in S33, a ratio R (=the number of ID-1/(the number of ID-1+the number of ID-2)) is calculated for each of the sequence sets. Thereafter, in S34, for each sequence set, a magnitude relationship between the ratio R and the threshold value is determined by comparing the threshold value set in advance. In S35, each sample is identified for the presence or absence of a disease based on the number of sequence sets showing a ratio R greater than the threshold value.

Here, RNA decoding may be performed, for example, by techniques for sequencing such as next generation sequencer (NGS), nanopore sequencer, Sanger method (electrophoresis, capillary sequencer), and Maxam Gilbert method. Alternatively, at least two of these methods may be used in combination.

According to the second embodiment, a method capable of identifying a sample derived from a patient with higher accuracy is provided.

### Third Embodiment

A third embodiment is a candidate sequence of a biomarker for identifying a sample derived from a cancer patient. Here, the biomarker is also simply referred to as a marker. Examples of the candidate sequence for a biomarker are shown in Table 3.

TABLE 3

| Marker | First reference | | Second reference | |
|---|---|---|---|---|
| set | sequence | | sequence | |
| number | (5′ → 3′) | SEQ ID NO: | (5′→3′) | SEQ ID NO: |
| Set 1 | ATGACCTATGAATTGACAGAC | SEQ ID NO: 6 | CTGACCTATGAATTGACAGCC | SEQ ID NO: 14 |
| Set 2 | CAAAGTGCTTACAGTGCAGGTAG | SEQ ID NO: 7 | CAAAGTGCTCATAGTGCAGGTAG | SEQ ID NO: 15 |
| Set 3 | TAGCACCATCTGAAATCGGTTA | SEQ ID NO: 8 | TAGCACCATTTGAAATCGGTTA | SEQ ID NO: 16 |
| Set 4 | TGGAATGTAAAGAAGTATGTAT | SEQ ID NO: 1 | TGGAATGTAAGGAAGTATGTAT | SEQ ID NO: 2 |
| Set 5 | TAGCAGCACATCATGGTTTACA | SEQ ID NO: 9 | TAGCAGCACATAATGGTTTGTG | SEQ ID NO: 17 |
| Set 6 | TGAGGTAGTAGGTTGTATGGTT | SEQ ID NO: 10 | TGAGGTAGTAGGTTGTGTGGTT | SEQ ID NO: 18 |
| Set 7 | CTTTCAGTCGGATGTTTACAGC | SEQ ID NO: 11 | CTTTCAGTCGGATGTTTGCAGC | SEQ ID NO: 19 |
| Set 8 | TTCACAGTGGCTAAGTTCCGC | SEQ ID NO: 12 | TTCACAGTGGCTAAGTTCTGC | SEQ ID NO: 20 |
| Set 9 | TGGAATGTAAAGAAGTATGTAT | SEQ ID NO: 13 | TGGAATGTAAAGAAGTATGAAT | SEQ ID NO: 21 |
| | | | TGGAATGTAAAGAAGTATGGAT | SEQ ID NO: 22 |
| | | | TGGAATGTAAAGAAGTATGCAT | SEQ ID NO: 23 |

The candidate sequences for a biomarker shown in Table 3 can be used as a sequence set including a first reference sequence and a second reference sequence. The underlined portion is a single base substitution site. The respective sequences are full-length sequences of the first reference sequence or the second reference sequence, respectively. The reference sequence to be counted as the first short-chain nucleic acid (ID-2) may be a full-length sequence or any sequence in which the 3′-end is deleted from the full-length sequence, or sequences of all lengths obtained by sequentially deleting the full-length sequence and the 3′-end, that is, all sequence groups having different lengths from each other. The sequence in which the 3′-end is deleted is a sequence having a length of at least 10 bases or more, which is completely matched from the 5′-end of the full-length sequence and has a single base substitution site. The same applies to the second short-chain nucleic acid (ID-1). Table 4 shows an example of a sequence group in a case where 9 types of sequences for use as biomarkers are used in combination as a reference sequence.

The ID attached to each set of an example of the sequence group is indicated by “ID-2” as the first reference sequence and “ID-1” as the second reference sequence, and is indicated by the “serial number at the time of experiment”, “type of base at the substitution site”, and “the number of deleted bases at 3′-end” for each set. For example, in the case of the first reference sequence in set 1, it is “10_C_−9”, the reference number at the time of experiment is No. 10, the base at the substitution site is cytosine (C), and the number of deletions at the 3′-end is −9.

The type of the base of the single base substitution site in the second reference sequence may be one corresponding base or all 3 types of corresponding bases as shown in set 9. In this case, the sequence counted as (the number of ID-1+the number of ID-2) is targeted for all the 4 types of bases (all). If desired, all 3 types of sets 1 to 8 may also be used. Alternatively, any one base may be selected for all sets.

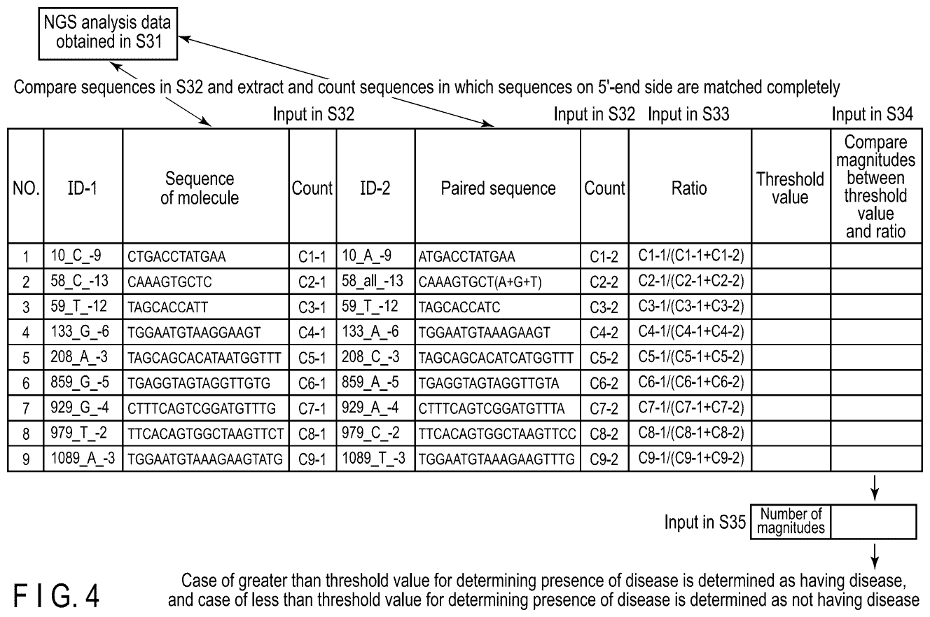

FIG. 4 illustrates an example of data processing flow when the above procedure using 9 types of marker sets is adapted for the method of the second embodiment. This is an example of outputting data obtained in each step as a table. “Sequence of molecule” and “paired sequence” are output from the data of the NGS analysis obtained for the nucleic acid present in the sample in S31 while referring to the sequences of used marker set numbers 1 to 9. In S32, the number of first short-chain nucleic acids (number of ID-2) and the number of second short-chain nucleic acids (number of ID-1) are counted and output to the respective “count” columns. In S33, the ratio R is calculated for each corresponding sequence and output to the column of “ratio”. The “threshold value” for each marker is input in advance or output to each column based on a test performed in advance. In S34, as a result of comparing each ratio with the threshold value, a marker whose ratio is greater than the threshold value is checked off in the column of “compare magnitudes between threshold value and ratio”. In S35, the number of markers whose ratio is determined to be greater than the threshold value in S34 is output. By comparing the number of markers with a predetermined threshold value, a sample is identified for the presence or absence of a disease or a magnitude of risk.

The reference sequences of set Nos. 1 to 9 shown in Table 4 can identify a sample derived from a cancer patient as a marker. In addition, it is possible to further improve the accuracy by simultaneously using all the 9 types of markers as a marker group in the sample identification method.

### Fourth Embodiment

A fourth embodiment is a biomarker for identifying a sample derived from a cancer patient. Examples of the biomarker are shown in Tables 5 to 13. In Tables 5 to 13, as a marker set selected from the sets 1 to 9 of marker candidates shown in Table 3, sequences to be indexes for all sequences to be counted as the first short-chain nucleic acid and the second short-chain nucleic acid are listed. The sequence in each table is a sequence in which pancreatic cancer can be identified at AUC>0.7 from the sequence group in which the 3′ side of the sequence of the marker candidate shown in Table 3 is deleted. Tables are listed in the order of decreasing by one base from the top to the bottom. The marker candidates shown in Table 3 are full-length sequences of the respective reference sequences. The number of deletions shown in each one of Tables 5 to 13 indicates the number of deletions from the sequence shown in Table 3.

For the bases of the single base substitution sites to be used for the calculation of the ratio R, the column “Calculation Formula” is referred to. “all” is calculated using the wild-type base and the other 3 types of bases. In the column of “AUC”, the Western calendar of the year in which the sample was analyzed was described. In each table, all sequences that can be used as the second reference sequence are described in the order in which the sequence is shortened by one base from the top to the bottom. The first reference sequence is a sequence in which the bases of the second reference sequence (ID-1) are substituted at positions counted from the 5′-end by the numbers listed in the column “position” with the base described in the column “WT”. In the table, “WT” represents a wild-type base, and “mu” represents a mutant-type base. Here, in marker set 2, only a sequence in which 13 bases were deleted from the full-length sequence of the reference sequence shown in Table 3 was shown. A standard sequence to be counted may be used as the second short-chain nucleic acid (ID-1), and at least one sequence described in Tables 5 to 13 may be used as an index sequence. In addition, the sequences described in any one or more of Tables 5 to 13 may be used in combination and simultaneously, one or more sequences described in any one or more of the tables may be used in combination and simultaneously, or one or more sequences described in all of Tables 5 to 13 may be used in combination and simultaneously. Alternatively, all sequences listed in all Tables 5 to 13 may be used in combination and simultaneously. In addition, as the marker set, at least two or more kinds of one short-chain sequence set selected from the sequence sets shown in Tables 5 to 13 or one sequence selected from the sequence sets shown in Tables 5 to 13 may be used in combination. This makes it possible to identify a sample derived from a cancer patient with high accuracy. The cancer identified here is, for example, pancreatic cancer.

TABLE 5

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 1 | 10_C_-2 | CTGACCTATGAATTGACAG | 45 | C/(A + C) | 1 | A | C | 19 | 0.812 | 0.786 | 0.844 |
| 1 | 10_C_-3 | CTGACCTATGAATTGACA | 46 | C/(A + C) | 1 | A | C | 18 | 0.813 | 0.786 | 0.845 |
| 1 | 10_C_-4 | CTGACCTATGAATTGAC | 47 | C/(A + C) | 1 | A | C | 17 | 0.812 | 0.786 | 0.844 |
| 1 | 10_C_-5 | CTGACCTATGAATTGA | 48 | C/(A + C) | 1 | A | C | 16 | 0.813 | 0.786 | 0.845 |
| 1 | 10_C_-6 | CTGACCTATGAATTG | 49 | C/(A + C) | 1 | A | C | 15 | 0.813 | 0.786 | 0.845 |
| 1 | 10_C_-_7 | CTGACCTATGAATT | 50 | C/(A + C) | 1 | A | C | 14 | 0.813 | 0.786 | 0.845 |
| 1 | 10_C_-8 | CTGACCTATGAAT | 51 | C/(A + C) | 1 | A | C | 13 | 0.814 | 0.786 | 0.847 |
| 1 | 10_C_-9 | CTGACCTATGAA | 27 | C/(A + C) | 1 | A | C | 12 | 0.814 | 0.786 | 0.849 |
| 1 | 10_C_-10 | CTGACCTATGA | 52 | C/(A + C) | 1 | A | C | 11 | 0.815 | 0.787 | 0.849 |
| 1 | 10_C_-11 | CTGACCTATG | 53 | C/(A + C) | 1 | A | C | 10 | 0.815 | 0.787 | 0.849 |
| 1 | 10_C_-2 | CTGACCTATGAATTGACAG | 45 | C/all | 1 | A | C | 19 | 0.809 | 0.786 | 0.834 |
| 1 | 10_C_-3 | CTGACCTATGAATTGACA | 46 | C/all | 1 | A | C | 18 | 0.809 | 0.786 | 0.835 |
| 1 | 10_C_-4 | CTGACCTATGAATTGAC | 47 | C/all | 1 | A | C | 17 | 0.810 | 0.787 | 0.835 |
| 1 | 10_C_-5 | CTGACCTATGAATTGA | 48 | C/all | 1 | A | C | 16 | 0.811 | 0.788 | 0.835 |
| 1 | 10_C_-6 | CTGACCTATGAATTG | 49 | C/all | 1 | A | C | 15 | 0.810 | 0.787 | 0.835 |
| 1 | 10_C_-7 | CTGACCTATGAATT | 50 | C/all | 1 | A | C | 14 | 0.810 | 0.787 | 0.835 |
| 1 | 10_C_-8 | CTGACCTATGAAT | 51 | C/all | 1 | A | C | 13 | 0.811 | 0.790 | 0.835 |
| 1 | 10_C_-_9 | CTGACCTATGAA | 27 | C/all | 1 | A | C | 12 | 0.811 | 0.789 | 0.837 |
| 1 | 10_C_-10 | CTGACCTATGA | 52 | C/all | 1 | A | C | 11 | 0.811 | 0.790 | 0.837 |
| 1 | 10_C_-11 | CTGACCTATG | 53 | C/all | 1 | A | C | 10 | 0.811 | 0.790 | 0.837 |

TABLE 6

| | | Sequence | SEQ | Calcula- | | | | | AUC | | |
|---|---|---|---|---|---|---|---|---|---|---|---|
| | | of | ID | tion | Posi- | | | | | 2021 + | |
| No. | ID-1 | molecule | NO: | formula | tion | WT | mu | Length | all | 2020 | 2022 |
| 2 | 58_C_-13 | CAAAGTGCTC | 28 | C/all | 10 | T | C | 10 | 0.859 | 0.874 | 0.786 |

TABLE 7

| | | | | Calcula- | | | | | AUC | | |
|---|---|---|---|---|---|---|---|---|---|---|---|
| | | Sequence of | SEQ | tion | Posi- | | | | | 2021 + | |
| No. | ID-1 | molecule | ID NO: | formula | tion | WT | mu | Length | all | 2020 | 2022 |
| 3 | 59_T_-1 | TAGCACCATTTGAAATCGGTT | 54 | T/(C + T) | 10 | C | T | 21 | 0.764 | 0.769 | 0.724 |
| 3 | 59_T_-2 | TAGCACCATTTGAAATCGGT | 55 | T/(C + T) | 10 | C | T | 20 | 0.746 | 0.717 | 0.743 |
| 3 | 59_T_-3 | TAGCACCATTTGAAATCGG | 56 | T/(C + T) | 10 | C | T | 19 | 0.746 | 0.722 | 0.733 |
| 3 | 59_T_-4 | TAGCACCATTIGAAATCG | 57 | T/(C + T) | 10 | C | T | 18 | 0.748 | 0.723 | 0.733 |
| 3 | 59_T_-5 | TAGCACCATTTGAAATC | 58 | T/(C + T) | 10 | C | T | 17 | 0.744 | 0.729 | 0.717 |
| 3 | 59_T_-6 | TAGCACCATTTGAAAT | 59 | T/(C + T) | 10 | C | T | 16 | 0.745 | 0.728 | 0.720 |
| 3 | 59_T_-7 | TAGCACCATTTGAAA | 60 | T/(C + T) | 10 | C | T | 15 | 0. 744 | 0.728 | 0.715 |
| 3 | 59_T_-8 | TAGCACCATTTGAA | 61 | T/(C + T) | 10 | C | T | 14 | 0.746 | 0.730 | 0.719 |
| 3 | 59_T_-9 | TAGCACCATTTGA | 62 | T/(C + T) | 10 | C | T | 13 | 0.746 | 0.729 | 0.719 |
| 3 | 59_T_-10 | TAGCACCATTTG | 63 | T/(C + T) | 10 | C | T | 12 | 0.746 | 0.731 | 0.717 |
| 3 | 59_T_-11 | TAGCACCATTT | 64 | T/(C + T) | 10 | C | T | 11 | 0.747 | 0.730 | 0.720 |
| 3 | 59_T_-12 | TAGCACCATT | 29 | T/(C + T) | 10 | C | T | 10 | 0.745 | 0.727 | 0.720 |
| 3 | 59_T_-1 | TAGCACCATTTGAAATCGGTT | 54 | T/all | 10 | C | T | 21 | 0.764 | 0.768 | 0.724 |
| 3 | 59_T_-2 | TAGCACCATTTGAAATCGGT | 55 | T/all | 10 | C | T | 20 | 0.746 | 0.716 | 0.743 |
| 3 | 59_T_-3 | TAGCACCATTTGAAATCGG | 56 | T/all | 10 | C | T | 19 | 0.746 | 0.722 | 0.733 |
| 3 | 59_T_-4 | TAGCACCATTTGAAATCG | 57 | T/all | 10 | C | T | 18 | 0.748 | 0.723 | 0.734 |
| 3 | 59_T_-5 | TAGCACCATTTGAAATC | 58 | T/all | 10 | C | T | 17 | 0.744 | 0.729 | 0.717 |
| 3 | 59_T_-6 | TAGCACCATTTGAAAT | 59 | T/all | 10 | C | T | 16 | 0.745 | 0.729 | 0.720 |
| 3 | 59_T_-7 | TAGCACCATTTGAAA | 60 | T/all | 10 | C | T | 15 | 0.744 | 0.729 | 0.717 |
| 3 | 59_T_-8 | TAGCACCATTTGAA | 61 | T/all | 10 | C | T | 14 | 0.746 | 0.731 | 0.719 |
| 3 | 59_T_-9 | TAGCACCATTTGA | 62 | T/all | 10 | C | T | 13 | 0.745 | 0.729 | 0.719 |
| 3 | 59_T_-10 | TAGCACCATTTG | 63 | T/all | 10 | C | T | 12 | 0.746 | 0.730 | 0.717 |
| 3 | 59_T_-11 | TAGCACCATTT | 64 | T/all | 10 | C | T | 11 | 0.746 | 0.728 | 0.720 |
| 3 | 59_T_-12 | TAGCACCATT | 29 | T/all | 10 | C | T | 10 | 0.745 | 0.727 | 0.720 |

TABLE 8

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 4 | 133_G_-6 | TGGAATGTAAGGAAGT | 30 | G/(A + G) | 11 | A | G | 16 | 0.788 | 0.835 | 0.737 |
| 4 | 133_G_-7 | TGGAATGTAAGGAAG | 65 | G/(A + G) | 11 | A | G | 15 | 0.789 | 0.834 | 0.739 |
| 4 | 133_G_-8 | TGGAATGTAAGGAA | 66 | G/(A + G) | 11 | A | G | 14 | 0.789 | 0.836 | 0.739 |
| 4 | 133_G_-9 | TGGAATGTAAGGA | 67 | G/(A + G) | 11 | A | G | 13 | 0.777 | 0.815 | 0.739 |
| 4 | 133_G_-10 | TGGAATGTAAGG | 68 | G/(A + G) | 11 | A | G | 12 | 0.777 | 0.815 | 0.739 |
| 4 | 133_G_-11 | TGGAATGTAAG | 26 | G/(A + G) | 11 | A | G | 11 | 0.776 | 0.814 | 0.739 |
| 4 | 133_G_-6 | TGGAATGTAAGGAAGT | 30 | G/all | 11 | A | G | 16 | 0.789 | 0.836 | 0.737 |
| 4 | 133_G_-7 | TGGAATGTAAGGAAG | 65 | G/all | 11 | A | G | 15 | 0.789 | 0.835 | 0.739 |
| 4 | 133_G_-8 | TGGAATGTAAGGAA | 66 | G/all | 11 | A | G | 14 | 0.789 | 0.837 | 0.739 |
| 4 | 133_G_-9 | TGGAATGTAAGGA | 67 | G/all | 11 | A | G | 13 | 0.777 | 0.816 | 0.739 |
| 4 | 133_G_-10 | TGGAATGTAAGG | 68 | G/all | 11 | A | G | 12 | 0.777 | 0.816 | 0.739 |
| 4 | 133_G_-11 | TGGAATGTAAG | 26 | G/all | 11 | A | G | 11 | 0.776 | 0.815 | 0.739 |

TABLE 9

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 5 | 208_A_-3 | TAGCAGCACATAATGGTTT | 31 | A/(C + A) | 12 | C | A | 19 | 0.892 | 0.897 | 0.882 |
| 5 | 208_A_-4 | TAGCAGCACATAATGGTT | 69 | A/(C + A) | 12 | C | A | 18 | 0.824 | 0.770 | 0.878 |
| 5 | 208_A_-5 | TAGCAGCACATAATGGT | 70 | A/(C + A) | 12 | C | A | 17 | 0.815 | 0.755 | 0.872 |
| 5 | 208_A_-6 | TAGCAGCACATAATGG | 71 | A/(C + A) | 12 | C | A | 16 | 0.828 | 0.782 | 0.877 |
| 5 | 208_A_-7 | TAGCAGCACATAATG | 72 | A/(C + A) | 12 | C | A | 15 | 0.828 | 0.782 | 0.878 |
| 5 | 208_A_-8 | TAGCAGCACATAAT | 73 | A/(C + A) | 12 | C | A | 14 | 0.828 | 0.782 | 0.878 |
| 5 | 208_A_-9 | TAGCAGCACATAA | 74 | A/(C + A) | 12 | C | A | 13 | 0.830 | 0.783 | 0.880 |
| 5 | 208_A_-10 | TAGCAGCACATA | 75 | A/(C + A) | 12 | C | A | 12 | 0.830 | 0.782 | 0.877 |
| 5 | 208_A_-3 | TAGCAGCACATAATGGTTT | 31 | A/all | 12 | C | A | 19 | 0.892 | 0.898 | 0.882 |
| 5 | 208_A_-4 | TAGCAGCACATAATGGTT | 69 | A/all | 12 | C | A | 18 | 0.824 | 0.771 | 0.878 |
| 5 | 208_A_-5 | TAGCAGCACATAATGGT | 70 | A/all | 12 | C | A | 17 | 0.815 | 0.775 | 0.872 |
| 5 | 208_A_-6 | TAGCAGCACATAATGG | 71 | A/all | 12 | C | A | 16 | 0.827 | 0.781 | 0.877 |
| 5 | 208_A_-7 | TAGCAGCACATAATG | 72 | A/all | 12 | C | A | 15 | 0.828 | 0.781 | 0.878 |
| 5 | 208_A_-8 | TAGCAGCACATAAT | 73 | A/all | 12 | C | A | 14 | 0.828 | 0.781 | 0.878 |
| 5 | 208_A_-9 | TAGCAGCACATAA | 74 | A/all | 12 | C | A | 13 | 0.830 | 0.783 | 0.880 |
| 5 | 208_A_-10 | TAGCAGCACATA | 75 | A/all | 12 | C | A | 12 | 0.829 | 0.780 | 0.877 |

TABLE 10

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 6 | 859_G_-4 | TGAGGTAGTAGGTTGTGT | 76 | G/(A + G) | 17 | A | G | 18 | 0.764 | 0.743 | 0.844 |
| 6 | 859_G_-5 | TGAGGTAGTAGGTTGTG | 32 | G/(A + G) | 17 | A | G | 17 | 0.764 | 0.743 | 0.844 |
| 6 | 859_G_-4 | TGAGGTAGTAGGTTGTGT | 76 | G/all | 17 | A | G | 18 | 0.764 | 0.743 | 0.844 |
| 6 | 859_G_-5 | TGAGGTAGTAGGTTGTG | 32 | G/all | 17 | A | G | 17 | 0.764 | 0.743 | 0.844 |

TABLE 11

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 7 | 929_G_-3 | CTTTCAGTCGGATGTTTGC | 77 | G/(A + G) | 18 | A | G | 19 | 0.748 | 0.789 | 0.703 |
| 7 | 929_G_-4 | CTTTCAGTCGGATGTTTG | 33 | G/(A + G) | 18 | A | G | 18 | 0.764 | 0.809 | 0.707 |
| 7 | 929_G_-3 | CTTTCAGTCGGATGTTTGC | 77 | G/all | 18 | A | G | 19 | 0.748 | 0.789 | 0.703 |
| 7 | 929_G_-4 | CTTTCAGTCGGATGTTTG | 33 | G/all | 18 | A | G | 18 | 0.763 | 0.809 | 0.707 |

TABLE 12

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 8 | 979_T_0 | TTCACAGTGGCTAAGTTCTGC | 20 | T/(C + T) | 19 | C | T | 21 | 0.864 | 0.915 | 0.776 |
| 8 | 979_T_-1 | TTCACAGTGGCTAAGTTCTG | 78 | T/(C + T) | 19 | C | T | 20 | 0.865 | 0.938 | 0.781 |
| 8 | 979_T_-2 | TTCACAGTGGCTAAGTTCT | 34 | T/(C + T) | 19 | C | T | 19 | 0.863 | 0.935 | 0.774 |
| 8 | 979_T_0 | TTCACAGTGGCTAAGTTCTGC | 20 | T/all | 19 | C | T | 21 | 0.863 | 0.913 | 0.776 |
| 8 | 979_T_-1 | TTCACAGTGGCTAAGTTCTG | 78 | T/all | 19 | C | T | 20 | 0.865 | 0.938 | 0.781 |
| 8 | 979_T_-2 | TTCACAGTGGCTAAGTTCT | 34 | T/all | 19 | C | T | 19 | 0.863 | 0.936 | 0.774 |

TABLE 13

| | | | SEQ | Calcula- | | | | | AUC | | |
|---|---|---|---|---|---|---|---|---|---|---|---|
| | | Sequence of | ID | tion | Posi- | | | | | 2021 + | |
| No. | ID-1 | molecule | NO: | formula | tion | WT | mu | Length | all | 2020 | 2022 |
| 9 | 1089_A_-3 | TGGAATGTAAAGAAGTATG | 35 | A/(T + A) | 17 | T | A | 19 | 0.749 | 0.755 | 0.734 |
| 9 | 1089_A_-4 | TGGAATGTAAAGAAGTAT | 79 | A/(T + A) | 17 | T | A | 18 | 0.731 | 0.719 | 0.735 |
| 9 | 1089_A_-5 | TGGAATGTAAAGAAGTA | 80 | A/(T + A) | 17 | T | A | 17 | 0.731 | 0.718 | 0.735 |
| 9 | 1089_A_-3 | TGGAATGTAAAGAAGTATG | 35 | A/all | 17 | T | A | 19 | 0.747 | 0.754 | 0.734 |
| 9 | 1089_A_-4 | TGGAATGTAAAGAAGTAT | 79 | A/all | 17 | T | A | 18 | 0.729 | 0.718 | 0.735 |
| 9 | 1089_A_-5 | TGGAATGTAAAGAAGTA | 80 | A/all | 17 | C | A | 17 | 0.728 | 0.717 | 0.731 |

According to the fourth embodiment, it is possible to identify a sample derived from a cancer patient with high accuracy. Furthermore, it is predicted that a remarkable effect is exerted also in cancer treatment and research.

### Experiment Part

### Example 1

The reference sequence was set using the specimens shown in Table 14.

TABLE 14
Specimen used

| | | Number of |
|---|---|---|
| Analysis | Specimen | specimens |
| 2020 | Pancreatic | 24 |
| | cancer | |
| 2021 | Non-cancer | 24 |
| | Pancreatic | 24 |
| | cancer | |
| 2022 | Non-cancer | 24 |
| | Pancreatic | 24 |
| | cancer | |

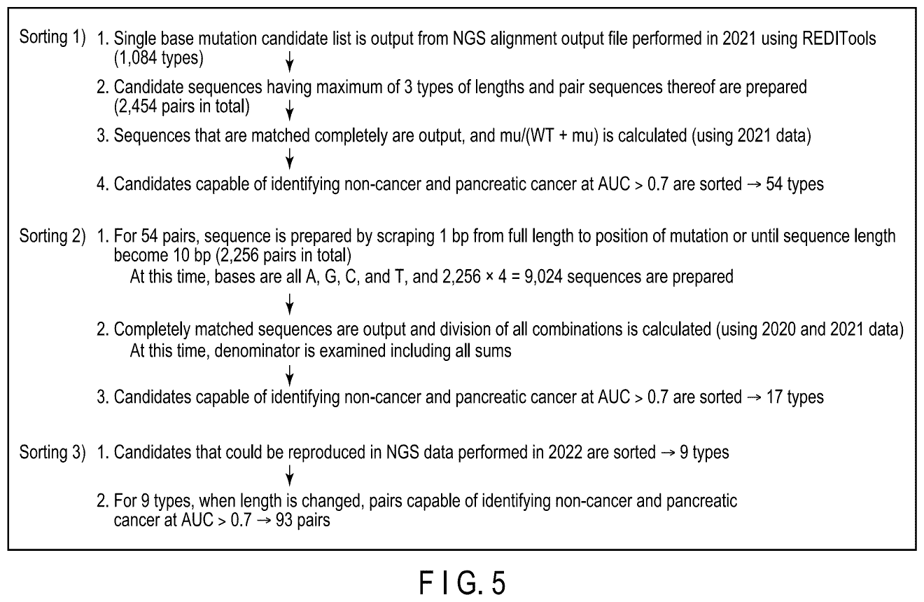

The obtained specimen samples are 24 specimens analyzed in 2020 from pancreatic cancer patients, 24 specimens from non-cancer subjects and 24 specimens from pancreatic cancer patients in 2021, 24 specimens from non-cancer subjects and 24 specimens from pancreatic cancer patients in 2022, that is, 120 specimens in total. Three stages of sorting were performed to set the reference sequence. The procedure is illustrated in FIG. 5.

First, in sorting 1), a single base mutation candidate list was output from the NGS alignment output file performed in 2021 using REDITools (1,084 types). Subsequently, candidate sequences having a maximum of 3 types of lengths and pair sequences thereof were prepared (2,454 pairs in total). Furthermore, sequences that were matched completely between the candidate sequences were output, and mu/(WT+mu) was calculated. At this time, data in 2021 was used as data. Then, candidates capable of identifying between non-cancer and pancreatic cancer at AUC>0.7 were sorted. 54 types of candidates were obtained in sorting 1.

Subsequently, in sorting 2), for the obtained 54 pairs, a sequence was prepared by deleting 1 bp from the full length to the position of mutation or until the sequence length became 10 bp (2,256 pairs in total). At this time, bases of single base substitution sites were assumed to be all A, G, C, and T, and 2,256×4=9,024 sequences were prepared. Next, the completely matched sequences were output and the division of all combinations was calculated. At this time, data in 2020 and 2021 was used as data. In addition, in the examination, the denominator was examined including all the sums. From the obtained results, candidates capable of identifying between non-cancer and pancreatic cancer at AUC>0.7 were sorted. 17 types of candidates were obtained in sorting 2.

Finally, sorting 3) was performed. In the NGS data performed in 2022, candidates whose results could be reproduced were sorted. As a result, 9 types of candidates were obtained in sorting 3. For these 9 types, when the length was changed, pairs in which non-cancer and pancreatic cancer could be identified at AUC>0.7 were obtained. The resulting sequences were 93 pairs. The sequences and data are shown in Tables 5 to 13.

As shown in Tables 5 to 13, any sequence can be used as a biomarker capable of identifying pancreatic cancer with high accuracy at AUC>0.7.

### Example 2

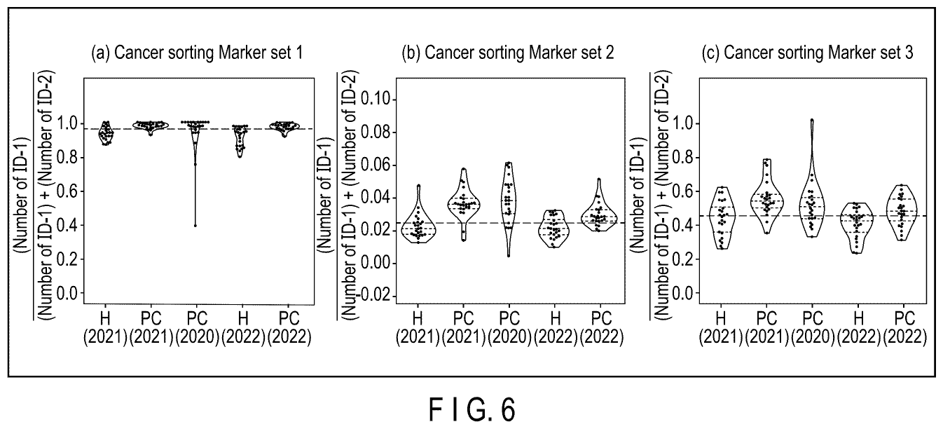

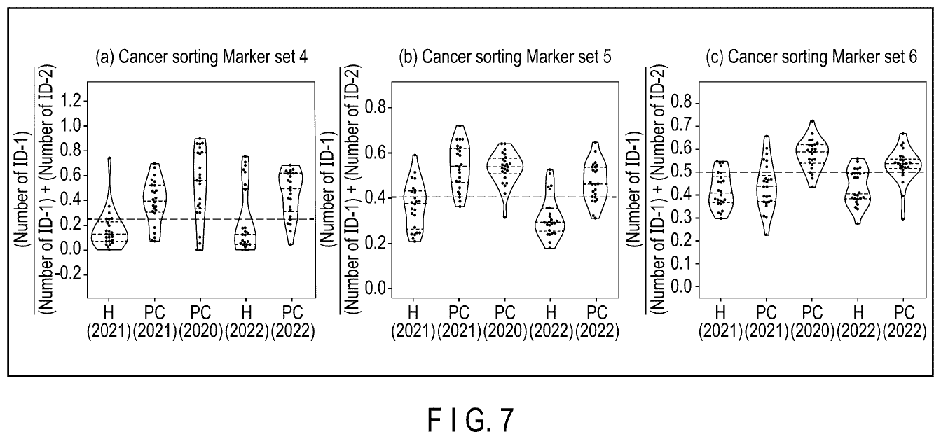

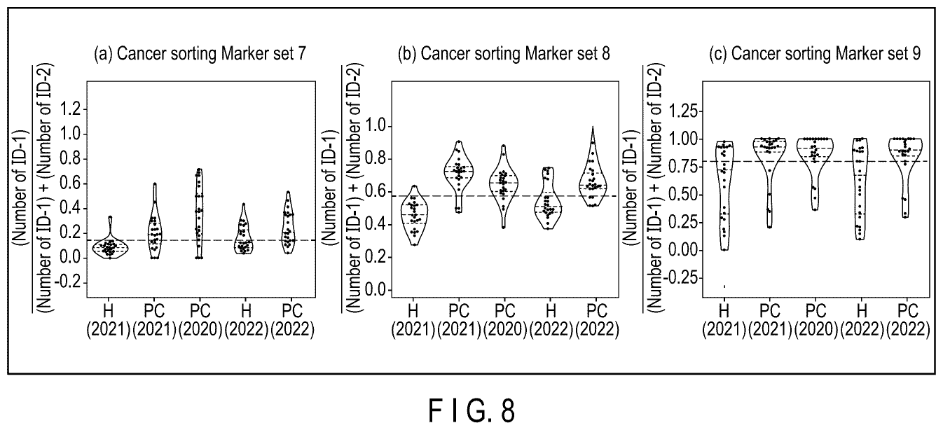

The results obtained by plotting the ratio R of each marker set shown in Tables 5 to 13 on a graph are illustrated in FIGS. 6 to 8. In each of the marker sets, the threshold values for identifying between non-cancer and pancreatic cancer were 0.96, 0.025, 0.46, 0.25, 0.4, 0.5, 0.14, 0.57, and 0.8 in the marker sets 1 to 9, respectively. There was a different tendency in the ratio R between non-cancer and pancreatic cancer.

### Example 3

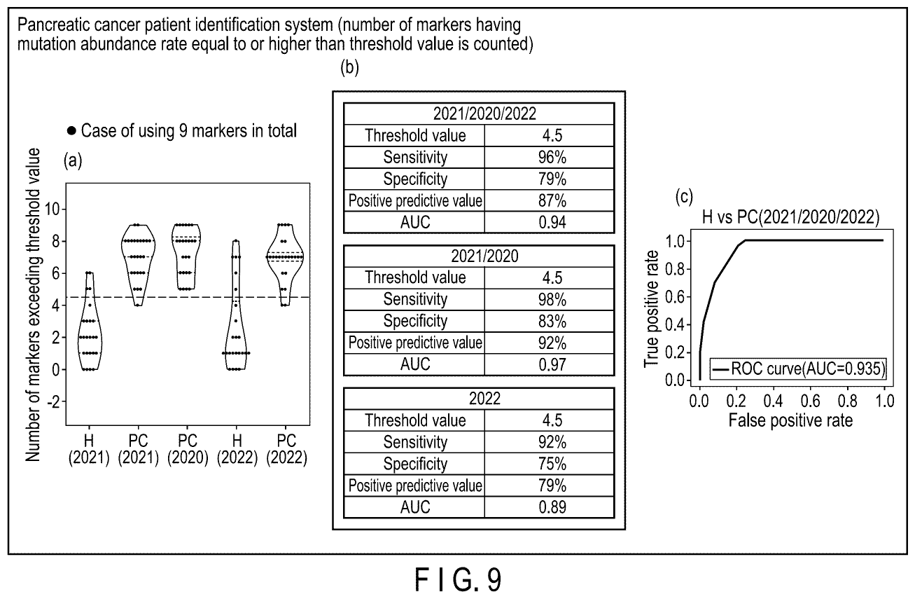

From each of the results obtained for the marker sets 1 to 9, the number of markers for which the ratio R exceeded the threshold value was counted and the results are illustrated in FIG. 9. The number of markers exceeding the threshold value for all 9 markers was shown for each type of and analyzed year of the subject from which the sample was derived. As illustrated in FIG. 9(a), the samples derived from the non-cancer subjects and the samples from the pancreatic cancer patient subjects showed more clearly different tendencies. As illustrated in FIG. 9(b), it is clear that samples can be identified with little variation in data analyzed in any year. As illustrated in FIG. 9(c), the AUC in the entire sample is 0.935, and it is clear that high accuracy can be obtained.

### Example 4

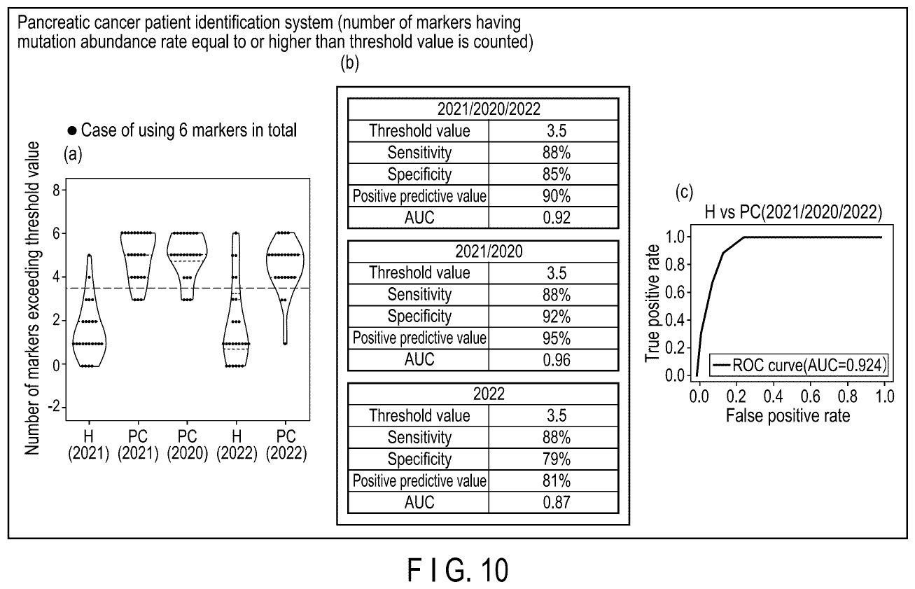

FIG. 10 illustrates the results using 6 markers. The 6 markers used are marker sets 3, 4, 5, 7, 8, and 9. This is a marker group excluding a marker having a low threshold value in Example 2 and having a possibility of causing an error in determination, and a marker having a sequence common to the let7-type miRNA in which a large number of miRNAs having high homology were present when used as a reference sequence.

The results of counting the number of markers exceeding the threshold value for these 6 markers are shown. The samples derived from the non-cancer patient subjects and the samples from the pancreatic cancer patient subjects showed more clearly different tendencies. As illustrated in FIG. 10(a), the samples derived from the non-cancer patient subjects and the samples from the pancreatic cancer patient subjects showed more clearly different tendencies. As illustrated in FIG. 10(b), it is clear that samples can be identified with little variation in data analyzed in any year. As illustrated in FIG. 10(c), the AUC in the entire sample is 0.924, and it is clear that high accuracy can be obtained.

### Example 5

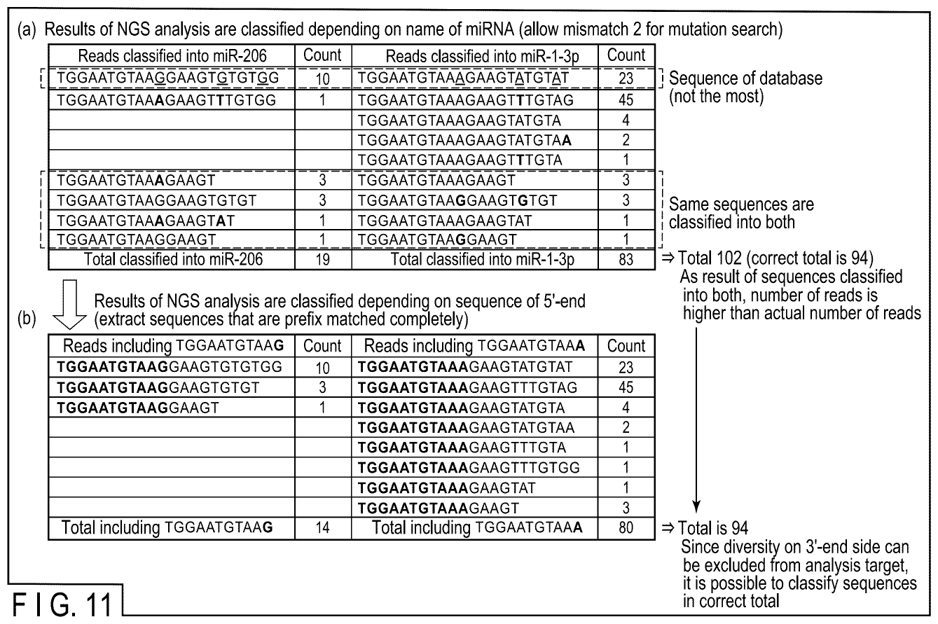

An image showing a comparison between the case of the selection of the short-chain nucleic acid to be analyzed was classified by the name of miRNA and the case of classification using a sequence that is prefix matched from the 5′-end of the sequence present in the sample was summarized in FIG. 11. FIG. 11(a) is an image of a result of NGS analysis performed by classification by name of miRNA. For mutation search, 2 mismatches are allowed and alignment is performed. Performing alignment while allowing mismatches is a general method in the case of analyzing sequences having diversity such as miRNAs. In this case, a sequence selected from the database is used as a marker. The sequences of the miRNAs are registered in the database under different names, but in practice, there may be only a few base differences. The miR-206 and the miR-1-3p shown as an example in FIG. 11(a) differ only in the underlined 3 bases, and the other sequences are common. When the NGS analysis result is aligned with respect to such a marker, a sequence classified into both miR-206 and miR-1-3p surrounded by a dotted line frame is generated. As a result, although there are originally only 94 sequences obtained from the NGS analysis result of the specimen, a result in which there are 102 sequences due to the presence of sequences classified into both is obtained. That is, ambiguity occurs in the result, leading to deterioration of reproducibility.

On the other hand, FIG. 11(b) illustrates a result of performing NGS analysis by the method according to the present embodiment. In the method according to the present embodiment, since only the sequence that is prefix matched with the sequence at the 5′-end is extracted from the sample nucleic acid, it is possible to exclude the diversity at the 3′-end from the analysis target. Therefore, it is possible to clearly classify close sequences, and it is possible to identify a sample with higher accuracy.

### Example 6

In the method according to the present embodiment, sequences that are completely matched from the 5′-end are used as markers, and it was examined how many bp the lengths of the sequence markers to be completely matched could be shortened. When the sequences of 2,656 types of miRNAs registered in miRBase (Release 22) were sequentially shortened by one base from the 3′-end, how many types of other miRNAs whose sequences from the 5′-end were completely matched were counted, and the sequence lengths that were matched with 10 or more types of miRNAs were confirmed.

TABLE 15

| | Number of target miRNAs | |
|---|---|---|
| Sequence | that match 10 or more | Proportion of |
| length | of types of miRNAs | 2,656 types |
| 12 | 10 | 0.4% |
| 11 | 13 | 0.5% |
| 10 | 17 | 0.6% |
| 9 | 21 | 0.8% |
| 8 | 24 | 0.9% |
| 7 | 94 | 3.5% |
| 6 | 428 | 16.1% |
| 5 | 1849 | 69.6% |
| 4 | 2630 | 99.0% |
| 3 | 2656 | 100.0% |

As a result, it is clear that when the reference sequence length is 8 bp, the other miRNAs whose sequences are completely matched are less than 1%, but when the reference sequence length is shortened to 6 bp, the sequence becomes identical to 10% or more of the miRNAs. The fact that the number of types of miRNAs having the same sequence increases means that the amount of the sequences having the sequence at the 5′-end changes depending on more factors, and thus, it is considered that stable quantification becomes difficult.

From the above results, a method for identifying a sample derived from a patient with high accuracy was provided.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

The correspondence between the sequence numbers and base sequences is summarized below:

SEQ ID NO: 1
TGGAATGTAAAGAAGTATGTAT
SEQ ID NO: 2
TGGAATGTAAGGAAGTATGTAT
SEQ ID NO: 3
TGGAATGTAAAGAAGTATGTA
SEQ ID NO: 4
TGGAATGTAAAGAAGTATGT
SEQ ID NO: 5
TGGAATGTAAA
SEQ ID NO: 6
ATGACCTATGAATTGACAGAC
SEQ ID NO: 7
CAAAGTGCTTACAGTGCAGGTAG
SEQ ID NO: 8
TAGCACCATCTGAAATCGGTTA
SEQ ID NO: 9
TAGCAGCACATCATGGTTTACA
SEQ ID NO: 10
TGAGGTAGTAGGTTGTATGGTT
SEQ ID NO: 11
CTTTCAGTCGGATGTTTACAGC
SEQ ID NO: 12
TTCACAGTGGCTAAGTTCCGC
SEQ ID NO: 13
TGGAATGTAAAGAAGTATGTAT
SEQ ID NO: 14
CTGACCTATGAATTGACAGAC
SEQ ID NO: 15
CAAAGTGCTCACAGTGCAGGTAG
SEQ ID NO: 16
TAGCACCATTTGAAATCGGTTA
SEQ ID NO: 17
TAGCAGCACATAATGGTTTACA
SEQ ID NO: 18
TGAGGTAGTAGGTTGTGTGGTT
SEQ ID NO: 19
CTTTCAGTCGGATGTTTGCAGC
SEQ ID NO: 20
TTCACAGTGGCTAAGTTCTGC
SEQ ID NO: 21
TGGAATGTAAAGAAGTATGAAT
SEQ ID NO: 22
TGGAATGTAAAGAAGTATGGAT
SEQ ID NO: 23
TGGAATGTAAAGAAGTATGCAT
SEQ ID NO: 24
TGGAATGTAAAGAAGTA
SEQ ID NO: 25
TGGAATGTAAGGAAGTA
SEQ ID NO: 26
TGGAATGTAAG
SEQ ID NO: 27
CTGACCTATGAA
SEQ ID NO: 28
CAAAGTGCTC
SEQ ID NO: 29
TAGCACCATT
SEQ ID NO: 30
TGGAATGTAAGGAAGT
SEQ ID NO: 31
TAGCAGCACATAATGGTTT
SEQ ID NO: 32
TGAGGTAGTAGGTTGTG
SEQ ID NO: 33
CTTTCAGTCGGATGTTTG
SEQ ID NO: 34
TTCACAGTGGCTAAGTTCT
SEQ ID NO: 35
TGGAATGTAAAGAAGTATG
SEQ ID NO: 36
ATGACCTATGAA
SEQ ID NO: 37
CAAAGTGCT (A + G + T)
SEQ ID NO: 38
TAGCACCATC
SEQ ID NO: 39
TGGAATGTAAAGAAGT
SEQ ID NO: 40
TAGCAGCACATCATGGTTT
SEQ ID NO: 41
TGAGGTAGTAGGTTGTA
SEQ ID NO: 42
CTTTCAGTCGGATGTTTA
SEQ ID NO: 43
TTCACAGTGGCTAAGTTCC
SEQ ID NO: 44
TGGAATGTAAAGAAGTTTG
SEQ ID NO: 45
CTGACCTATGAATTGACAG
SEQ ID NO: 46
CTGACCTATGAATTGACA
SEQ ID NO: 47
CTGACCTATGAATTGAC
SEQ ID NO: 48
CTGACCTATGAATTGA
SEQ ID NO: 49
CTGACCTATGAATTG
SEQ ID NO: 50
CTGACCTATGAATT
SEQ ID NO: 51
CTGACCTATGAAT
SEQ ID NO: 52
CTGACCTATGA
SEQ ID NO: 53
CTGACCTATG
SEQ ID NO: 54
TAGCACCATTTGAAATCGGTT
SEQ ID NO: 55
TAGCACCATTTGAAATCGGT
SEQ ID NO: 56
TAGCACCATTTGAAATCGG
SEQ ID NO: 57
TAGCACCATTTGAAATCG
SEQ ID NO: 58
TAGCACCATTTGAAATC
SEQ ID NO: 59
TAGCACCATTTGAAAT
SEQ ID NO: 60
TAGCACCATTTGAAA
SEQ ID NO: 61
TAGCACCATTTGAA
SEQ ID NO: 62
TAGCACCATTTGA
SEQ ID NO: 63
TAGCACCATTTG
SEQ ID NO: 64
TAGCACCATTT
SEQ ID NO: 65
TGGAATGTAAGGAAG
SEQ ID NO: 66
TGGAATGTAAGGAA
SEQ ID NO: 67
TGGAATGTAAGGA
SEQ ID NO: 68
TGGAATGTAAGG
SEQ ID NO: 69
TAGCAGCACATAATGGTT
SEQ ID NO: 70
TAGCAGCACATAATGGT
SEQ ID NO: 71
TAGCAGCACATAATGG
SEQ ID NO: 72
TAGCAGCACATAATG
SEQ ID NO: 73
TAGCAGCACATAAT
SEQ ID NO: 74
TAGCAGCACATAA
SEQ ID NO: 75
TAGCAGCACATA
SEQ ID NO: 76
TGAGGTAGTAGGTTGTGT
SEQ ID NO: 77
CTTTCAGTCGGATGTTTGC
SEQ ID NO: 78
TTCACAGTGGCTAAGTTCTG
SEQ ID NO: 79
TGGAATGTAAAGAAGTAT
SEQ ID NO: 80
TGGAATGTAAAGAAGTA.

What is claimed is:

1. A method for identifying a sample, the method comprising:
setting a set or a plurality of sets of a first reference sequence that includes a first sequence having a single base substitution site that is suggested to have a relationship with a target disease based on a predetermined criterion, the single base substitution site being a first base, and a second reference sequence that corresponds to the first reference sequence, includes the first sequence, and contains a second base having the single base substitution site that is different from the first base;
outputting, with respect to a short-chain nucleic acid group contained in the sample,
the number of first short-chain nucleic acids (the number of ID-2) having a sequence having a length of 10 bases or more and having the single base substitution site so as to be prefix matched with the first reference sequence from the 5′-end thereof, and
the number of second short-chain nucleic acids (the number of ID-1) having a sequence having a length of 10 bases or more and having the single base substitution site so as to be prefix matched with the second reference sequence from the 5′-end thereof;
calculating a ratio R=(the number of ID-1/(the number of ID-1+the number of ID-2)) for each corresponding sequence;
comparing a value of the ratio R with a predetermined threshold value to obtain a magnitude relationship between the value of the ratio R and the predetermined threshold value; and
determining, from the number of sequences in which the value of the ratio R is greater than the threshold value, whether or not a subject from whom the sample is derived has the disease or at risk of developing the disease.

2. The method according to claim 1, wherein the first reference sequence is a sequence set selected from the sequence sets shown in Tables 5 to 13, or at least two types of sequence sets each selected from the sequence sets shown in Tables 5 to 13 are used in combination.

3. The method according to claim 1, wherein the disease is cancer.

4. The method according to claim 1, wherein the disease is pancreatic cancer.

5. The method according to claim 1, wherein the short-chain nucleic acid is miRNA.

6. The method according to claim 1, wherein the sample is serum or plasma.

7. The method according to claim 1, wherein the method is performed by using a sequencing technique such as next generation sequencer, nanopore sequencer, Sanger method (electrophoresis, capillary sequencer), or Maxam Gilbert method.

8. A marker for identifying a sample as to whether or not the subject from whom the sample is derived has a disease, the marker being a biomarker, wherein the marker is a sequence set selected from the sequence sets shown in Tables 5 to 13, or a combination of at least two types of sequence sets each selected from the sequence sets shown in Tables 5 to 13.

9. The marker according to claim 8, wherein the disease of the subject is cancer.

10. The marker according to claim 8, wherein the disease of the subject is pancreatic cancer.
